(19) United States
(12) Patent Application Publication
(10) Pub. No.: US 2005/0153277 A1
(43) Pub. Date: Jul. 14, 2005

(54) Immunochemical assay device

(76) Inventors: Kai-Tsung Teng, Taipei (TW); Sheng-Wen Chang, Taipei (TW)

Correspondence Address: MORRISON & FOERSTER LLP, 755 PAGE MILL RD, PALO ALTO, CA, 94304-1018, US

(21) Appl. No.: 10/849,109

(22) Filed: May 18, 2004

(30) Foreign Application Priority Data
Jan. 13, 2004 (TW) 093100751

(51) Int. Cl. C12Q 1/70; C12M 1/34

(52) U.S. Cl. 435/5; 435/287.2

(57) ABSTRACT

An immunochemical assay device is proposed. The immunochemical assay device includes a base member, a liquid-flowing layer disposed on the base member and a light-permissible member attached on the liquid-flowing layer. A gap is interposed between the light-permissible member and the liquid-flowing layer, wherein at least an immobilized substance is disposed on the surface of the light-permissible member facing the liquid-flowing layer. The immobilized substance binds to a specific ligand-receptor complex contained in a liquid sample to form an assay marker after the liquid sample has been added into the gap. The liquid sample flows in a microfluidic environment formed between the light-permissible member and the liquid-flowing layer and reacts with the immobilized substance to produce a colored assay marker on the light-permissible member. As the light-permissible member provides better transmission of light than previously known paper materials do, the assay marker can be easily detected by human eyes.

### BACKGROUND OF THE INVENTION

1. Field of the Invention

The present invention relates to sample assay devices, and more particularly, to an immunochemical assay device with sample-flow through function for rapidly assaying a variety of biological samples.

2. Description of the Related Art

Rapid assay technologies are commonly applicable to many fields, such as food processing, agriculture, medicine, environmental protection and biology. Various methods of biological assays for detecting the presence of a specific substance such as enzymes, antibodies or pathogens (viruses or bacteria) in body fluid from humans or animals have been discovered. However, due to consideration of such issues as sensitivity, accuracy and operative effectiveness of assays, immunochemical detection is the most commonly used method among the present methods of biological assays.

In the so-called ‘sandwich’ method, immunochemical detection utilizes the specific binding between antigens and antibodies and between antigens and targets to sandwich an antigen (detection sample) between a labeled antibody and an antibody immobilized onto the surface of a solid support (detector disc). In the competition immunoassay method, an antibody bound to a solid support is exposed to a detection target and a labeled antigen. The amount of the labeled antigen bound on the solid surface is then determined to provide an indirect measurement of the amount of the detection target.

Immunochemical detection, regardless of the sandwich method or the competition method, has to be rapid, accurate and simple while requiring only a trace amount of samples (highly sensitive). In order to meet such requirements, immunoassay devices in solid phase have been developed to employ porous materials such as nylon, nitrocellulose, glass fibers or cellulose acetate as solid supports on which antigens, antibodies or detecting agents are blotted. Thus, detection samples are separated via siphonic phenomenon to cause the formation of colored products in such immunoassay.

Referring to FIG. 5 for illustrating U.S. Pat. No. 5,252,496 (“Carbon Black Immunochemical Label”) and U.S. Pat. No. 5,559,041 (“Immunoassay Devices and Materials”) applied by Princeton Biomedical Co. to disclose an immunochemical assay device which comprises a base member 10′ and an array disposed on the base member. The array comprises a reservoir pad 11′ to receive and contain a liquid sample; at least one filter zone 12′, separated and distinct from reservoir pad 11′ to exclude any unexpected substances or contaminants that degrade the color formation; a wicking membrane 13′ adjacent to the filter zone 12′ and disposed distally to reservoir pad 11′, the wicking membrane is made of paper or the liquid-absorptive material for absorbing a substantial amount of the sample that passes through the reservoir pad 11′ and the filter zone 12′; and an assay marker zone 16′ disposed on a pre-defined position of a surface of the wicking membrane 13′, wherein the reagent on the wicking membrane reacts with antigen or antibody bound to detection targets (such as specific pathogens) in the liquid sample, thereby indicating the results of detecting the samples via the color formation or fluorescent emission.

In reference to the above assay marker 16′, the reagent coated on the surface of wicking membrane 13′ makes the fabrication of immunochemical assay devices much easier. However, it has stringent criteria and only a few choices for the materials of the wicking membrane (nitrocellulose normally being used) as the reagent (primary antibodies normally being used) that provides the color formation or fluorescent emission is directly coated on the surface of the wicking membrane to allow efficient reaction with the reagent. Furthermore, while the wicking membrane is made of paper materials, factors such as weak tension and structure strength, contamination by hand touching or folding during the processing all make it difficult to transport the wicking membranes.

### SUMMARY OF THE INVENTION

The primary objective of the present invention is to provide an immunochemical assay device to promote a visualizing effect of a colored band, which can be easily detected by human eyes.

Another objective of the present invention is to provide an immunochemical assay device that allows a reduced quantity of reagents and samples required in a bioassay, so as to save on cost of making and using the immunochemical assay device.

A further objective of the present invention is to provide an immunochemical assay device with high sensitivity.

In accordance with the above and other objectives, the present invention proposes a novel immunochemical assay device which comprises a base member, a liquid-flowing layer disposed on the base member to receive and contain a liquid sample, and a light-permissible member attached on the liquid-flowing layer. A gap is interposed between the light-permissible member and the liquid-flowing layer to allow flow of the liquid sample, wherein at least one immobilized substance is disposed on a side of the light-permissible member facing the liquid-flowing layer to bind with a specific ligand-receptor complex contained in the liquid sample for forming an assay marker.

The above immunochemical assay device utilizes a microfluidic environment formed between a light-permissible member (e g. polystyrene transparent plastic sheetpiece) and a liquid-flowing layer to allow flow of the liquid sample prepared by mixing diluted testing samples (such as blood, urine or samples of other sources from patients) with reagents (such as secondary antibodies) in the microfluidic environment, so that the liquid sample reacts with the immobilized substance disposed on the light-permissible member. As the immobilized substance (such as primary antibodies) is bound to the surface of the light-permissible member using a spray-coating method, the immobilized substance binds to a specific ligand (detection targets such as specific pathogens or protein marker)-receptor (secondary antibodies) complex contained in the sample to form an assay marker. Then, the assay marker is attached to the light-permissible member to produce a colored effect, which is more easily detected by human eyes. While the light-permissible member and the liquid-flowing layer do not absorb the liquid, the liquid sample that stays between the light-permissible member and the liquid-flowing layer is not wasted and absorbed by paper material used in conventional immunoassay device. Thus, only a trace amount of samples is needed to achieve the color formation in the bioassay, minimizing the quantity of samples required.

### BRIEF DESCRIPTION OF THE DRAWINGS

The drawings included herein provide a further understanding of the present invention. A brief introduction of the drawings is as follows:

FIG. 1 is a schematic diagram of an immunochemical assay device proposed by the present invention.

FIG. 2 illustrates an immunochemical assay device with a cross-sectional view according to the present invention.

FIG. 3 is a brief diagram showing the process for detecting the reaction between a ligand-receptor complex in a liquid sample and an immobilized substance on a light-permissible member of an immunochemical assay device according to the present invention.

FIG. 4 illustrates an elevational view of a kit fabricated using immunochemical assay devices according to the present invention.

FIG. 5 illustrates a cross-sectional view of an immunoassay device disclosed in the prior-art.

### DETAILED DESCRIPTION OF THE EMBODIMENTS

The present invention is described in the following with specific embodiments, so that one skilled in the pertinent art can easily understand other advantages and effects of the present invention from the disclosure of the invention. The present invention may also be implemented and applied according to other embodiments, and the details may be modified based on different views and applications without departing from the spirit of the invention.

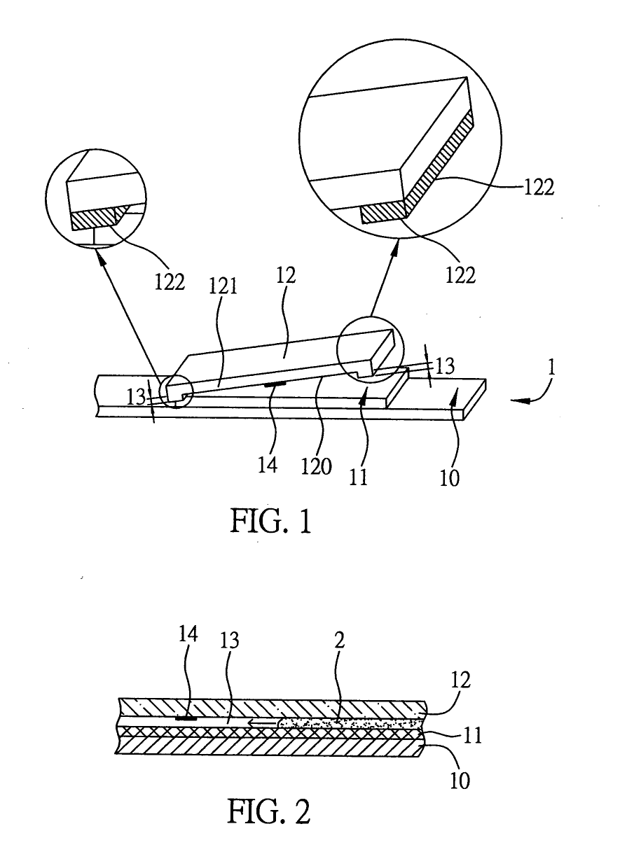

Referring to FIG. 1, the present invention provides an immunochemical assay device 1 which comprises a base member 10, a liquid-flowing layer 11 disposed on the base member, a light-permissible member 12 attached on the liquid-flowing layer and a gap 13 interposed between the light-permissible member and the liquid flowing layer. At least an immobilized substance 14 is disposed on a surface of the light-permissible member 12 facing the liquid-flowing layer 11. The immobilized substance 14 binds to a specific ligand-receptor complex (not shown) contained in a liquid sample to be detected (not shown) to form an assay marker (not shown) after the liquid sample has been added into the gap 13.

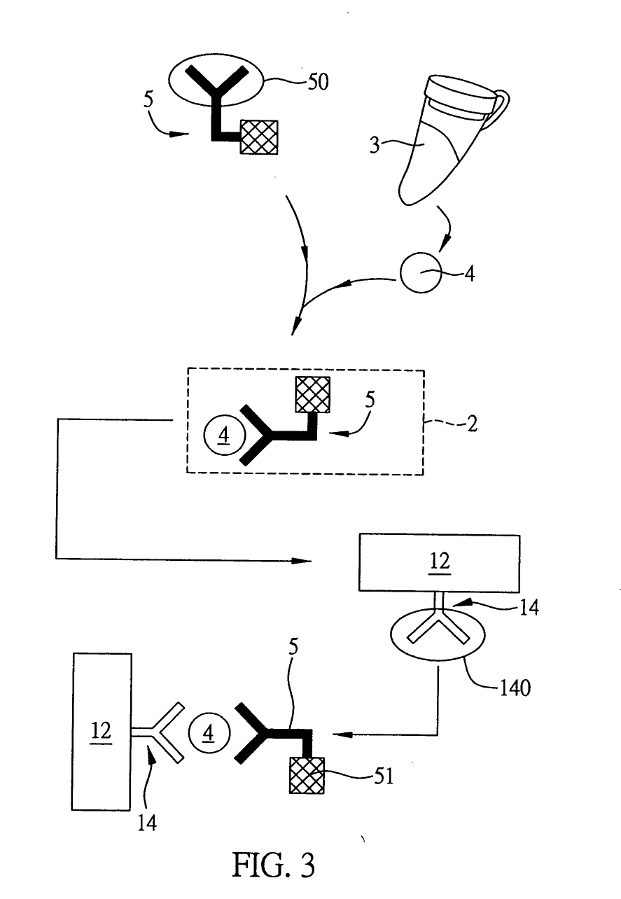

Again referring to FIG. 1, the light-permissible member 12 has one surface 120 facing the liquid-flowing layer 11, wherein the surface 120 is subjected to a conventional electrostatic treatment or film coating treatment before one or more detective bands made of at least the immobilized substance 14 can adhere to a pre-defined position. Moreover, the base member 10 and the liquid-flowing layer 11 can be fabricated using plastics, nitrocellulose, glass fibers, and other waterproof and non-liquid absorptive materials, and the light-permissible member 12 can be fabricated using glass or plastic materials such as polystyrene (PS) and acrylic. In this embodiment, light-permissible member 12 is a solid transparent plastic piece made of PS materials. The light-permissible member 12 comprises a sheet 121 and headrests 122, wherein the headrests 122 are formed to integrate on two ends of the sheet 121 to support the sheet 121 over the liquid-flowing layer 11. Thus, a gap 13 is interposed between the sheet 121 and the liquid-flowing layer 11 to allow the liquid sample (shown as numeral 2 in FIG. 2) to flow in the gap 13 in one direction (indicated by the arrow in FIG. 2).

Referring to FIG. 2, each of the headrests (not shown in the figure) has a thickness that determines a size of the gap 13. In this embodiment, the size smaller than 0.25 mm is preferred for the gap 13 and the immobilized substance 14 has a thickness far smaller than the thickness of the gap 13. And besides integrating the headrests 122 to the sheet 121 of the light-permissible member 12 described in this embodiment, the headrests 122 may also comprise various spacers attached between the light-permissible member 12 and the liquid-flowing layer 11 via adhesion or soldering, and such spacers including adhesive, paper, or plastic film. So, any element that is capable of supporting the light-permissible member 12 over the liquid-flowing layer 11 to form a gap smaller than 0.25 mm shall fall within the scope of the present invention.

The best mode for detecting the liquid sample with use of the immunochemical assay device of the present invention is described in the followings with the example of a bioassay card for detecting the SARS (Severe Acute Respiratory Syndrome) virus in the human body. It should be noted that the immunochemical assay device of this embodiment identifies whether the test subject is infected by the SARS virus, so the applications thereof are not limited as such. Therefore, applications in many fields such as food processing, agriculture, medicine and environmental-testing and any method that utilizes specific binding between antigens and antibodies according to the above immunochemical assay device for detecting the detection target are all encompassed in the scope of the present invention.

Referring to FIG. 3, a bioassay card is described according to another embodiment of the immunochemical assay device for detecting whether any SARS virus is present in human blood. In this embodiment, a blood sample 3 from a test subject (not shown) might contain a SARS virus 4 that serves as a ligand and a secondary antibody 5 that serves as a receptor for binding and labeling the SARS virus 4. A primary antibody 14 (as a primary antibody of this embodiment serves as the immobilized substance 14, the same label 14′ is used) that reacts with a SARS virus 4-Secondary antibody 5 complex to form a colored product, serves as an immobilized substance for the assay as demonstrated in FIG. 3.

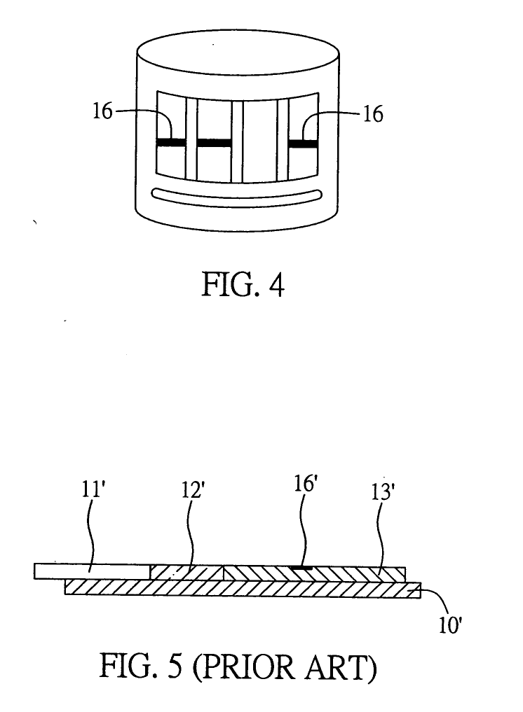

First, the blood sample 3 from the test subject is diluted 5000 times with a buffer solution and the diluted blood sample is added and mixed well with the secondary antibody 5 to prepare a liquid sample 2 as shown in FIG. 3. In this embodiment, an antigen-binding site 50 (Fragment Fab) of the secondary antibody 5 binds to the SARS virus 4 in the diluted blood sample to produce the SARS virus 4-Secondary antibody 5 complex if the test subject has been infected by the SARS virus 4. Then, the liquid sample 2 containing the SARS virus 4-Secondary antibody 5 complex is dropped onto a bioassay card 1 (The label ‘immunochemical assay device 1’ is used to indicate the immunochemical assay device represented by the bioassay card 1). When the liquid sample 2 flows through the gap (not shown) and makes contact with the primary antibody 14, an antigen binding site 140 of the primary antibody 14 binds to the other site of the SARS virus 4 and reacts with a chromophore 51 on the secondary antibody 5 to produce color if the SARS virus 4 is actually present in the liquid sample, thereby developing one or more color bands detected by human eyes (shown as numeral 16 in FIG. 4). In addition to colored bands developed via color formation, fluorescence, luminescence or other color development technologies can all be employed in the assay marker of this embodiment. However, the assay marker such as a colored band is attached to the light-permissible member 12 in the immunochemical assay device 1 proposed in the present invention, but not on previously known paper materials. Therefore, the colored band generated from the reaction makes the color formation more visible as the light-permissible member provides better light transmission than paper materials do.

The bioassay card of the above embodiment may be used in a single sheet or fabricated into a kit for detecting many samples in a parallel fashion. Referring to FIG. 4, any SARS virus present in samples from various sources of the test subject, such as blood, serum, urine or mucus from respiratory tract, can be detected at the same time, so that the result can be more easily compared or monitored by a clinician.

The immunochemical assay device proposed by the present invention utilizes a microfluidic environment formed between a light-permissible member (e g. polystyrene transmitted plastic sheet) and a liquid-flowing layer for detecting a liquid sample as a result of mixing diluted testing samples with secondary antibodies to flow in the microfluidic environment and react with a immobilized substance disposed on the light-permissible member. As the immobilized substance is bound to the surface of the light-permissible member by a spray-coating method, the immobilized substance binds to a specific ligand (detection target such as specific pathogens or protein marker)-receptor (secondary antibody in the reagent) complex contained in the sample to form an assay marker. Thus, the assay marker is attached to the light-permissible member to enhance visualization by human eyes. In addition, since the light-permissible member and the liquid-flowing layer do not absorb liquids, the liquid sample that stays between the light-permissible member and the liquid-flowing layer is not wasted and absorbed by paper materials used in a conventional immunoassay device. Thus, only a trace amount of samples would enable the color formation, further minimizing the amount of samples required for the assay.

It should be apparent to those skilled in the art that the above description is only illustrative of specific embodiments and examples of the present invention. The present invention should therefore cover various modifications and variations made to the herein-described structure and operations of the present invention, provided they fall within the scope of the present invention as defined in the following appended claims.

1. An immunochemical assay device, comprising:
a base member;
a liquid-flowing layer disposed on the base member; and
a light-permissible member attached on the liquid-flowing layer, wherein a gap is interposed between the light-permissible member and the liquid-flowing layer to allow flow of an assay liquid sample, and at least an immobilized substance is further disposed on a side of the light-permissible member facing the liquid-flowing layer for binding with a ligand-receptor complex contained in the assay liquid sample to form an assay marker.

2. The immunochemical assay device of claim 1, wherein the immunochemical assay device is a bioassay card.

3. The immunochemical assay device of claim 1, wherein the base member and the liquid-flowing layer are made of a material selected from the group consisting of plastics, nitrocellulose, glass fibers, and other materials non-absorptive to liquids.

4. The immunochemical assay device of claim 1, wherein the assay liquid sample comprises samples and at least one reagent.

5. The immunochemical assay device of claim 4, wherein the sample is diluted in serial dilutions.

6. The immunochemical assay device of claim 4, wherein the reagent comprises a secondary antibody serving as a receptor.

7. The immunochemical assay device of claim 6, wherein the secondary antibody carries at least one antigen binding site and one chromophore.

8. The immunochemical assay device of claim 1, wherein the light-permissible member is a solid plastic sheetpiece made of polystyrene (PS) material.

9. The immunochemical assay device of claim 1, wherein the light-permissible member comprises one sheet and a plurality of headrests, wherein each of the headrests is attached to one end of the sheet for supporting the sheet over the liquid-flowing layer.

10. The immunochemical assay device of claim 9, wherein the headrests are formed as an integral part of the sheet.

11. The immunochemical assay device of claim 9, wherein the headrest is made of materials selected from a group consisting of an adhesive, a paper, and a plastic film.

12. The immunochemical assay device of claim 1, wherein the gap has a height smaller than 0.25 mm.

13. The immunochemical assay device of claim 1, wherein the immobilized substance is a primary antibody.

14. The immunochemical assay device of claim 1, wherein the ligand is an assay target contained in the sample.

15. The immunochemical assay device of claim 14, wherein the target is a pathogen.

16. The immunochemical assay device of claim 14, wherein the target is a marker protein.

17. The immunochemical assay device of claim 14, wherein the target is a SARS virus.

18. The immunochemical assay device of claim 1, wherein the assay marker is a colored band.
